(19) United States
(12) Patent Application Publication
(10) Pub. No.: US 2005/0054999 A1
(43) Pub. Date: Mar. 10, 2005

(54) Nonwoven fabric laminate that reduces particle migration

(75) Inventors: Michael Tod Morman, Alpharetta, GA (US); Mark G. Kupelian, Atlanta, GA (US); Todd Sudduth, Cumming, GA (US); David Minh Tran, Neenah, WI (US); James Randall Morgan, Neenah, WI (US); Thomas Gerald Bolwerk, Appleton, WI (US); Hughey Kenneth Jeffries, Marietta, GA (US); Riley Kimbrough Middleton III, Lagrange, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/657,498

(22) Filed: Sep. 8, 2003

(51) Int. Cl. A61F 13/15; A61F 13/20

(52) U.S. Cl. 604/367

(57) ABSTRACT

The present invention provides a nonwoven fabric laminate that comprises a thin layer of fine fibers that has a basis weight of less than 1.5 grams per square meter. The present invention also provides disposable absorbent garments, such as diapers, that in such a nonwoven fabric laminate to reduce the migration of particles in absorbent garments.

### TECHNICAL FIELD

This invention relates to nonwoven fabric laminates and to absorbent articles such a diapers that include nonwoven fabric laminates.

### BACKGROUND

Personal care absorbent articles, such as disposable diapers, are typically configured to acquire and retain the body fluids for which the articles were designed, avoid excessive leakage of waste materials from the article and minimize the amount of any residue which migrates from the absorbent material onto the skin of a wearer. For example, diapers for infants are typically designed to accept large volumes of urine in multiple doses which can measure 60-100 milliliter per dose. Such diapers often require the use of high absorbency, superabsorbent particles to provide the needed absorbent capacity. Typically, superabsorbent gel particles are blended with woodpulp fibers to create an absorbent matrix. The matrix, however, is often unable to adequately contain the superabsorbent particles. As a result, dry particles can escape from the article prior to use, and wet particles can migrate from the absorbent matrix to the skin of the wearer. Although superabsorbent gel particles have not been observed to adverse affect skin health, the occurrence of foreign particles on the skin of an infant is not preferred by consumers and thus is not desirable. Accordingly, it would be desirable to produce personal care absorbent articles, particularly diapers, that reduce gel particle migration.

Conventional diapers include an absorbent portion that is disposed between a breathable, liquid impervious backing sheet that is also referred to as the outercover and a liquid pervious bodyside liner that contains the absorbent portion of the diaper from the wearer of the diaper. The bodyside liner that separates the wearer from the absorbent portion of the diaper must be liquid pervious so that the absorbent can absorb liquid waste. A single layer of spunbonded nonwoven fibers has been used as a body side liner in diapers because surfactant treated polypropylene spunbonded nonwoven fabrics are highly liquid pervious and inexpensive. However, spunbonded nonwoven fabrics consist of coarse spunbonded fibers that typically have diameters in the range of from about 8 to about 30 microns. Light weight spunbonded nonwoven fabrics used as diaper liners may have large pore sizes often in excess of ten times the spunbond fiber diameters. Dry superabsorbent gel particles can and frequently do migrate thorough spunbonded nonwoven webs. Wet superabsorbent particles may be pliable enough to go through smaller holes even when the particles are enlarged due to water absorption.

Conventional absorbent articles, such as those described above, have required more complicated manufacturing processes and more complex constructions to provide adequate performance. Despite the development of absorbent structures of the types surveyed above, there remains a need for absorbent structures which incorporate improved a component layer having a high resistance to the migration of particulate superabsorbent material as well as a high permeability to the passage of urine and other liquid body exudates. Attempts to reduce the migration of superabsorbent gel have employed various types of materials to shield the superabsorbent material from the wearer's skin. For example, barrier tissues and core wraps have been used to separate and contain the absorbent core portion of a diaper. U.S. Pat. No. 5,458,592 and EP 1073390 B1 describe thermoplastic fibrous tissues for wrapping absorbent cores that are referred to a core wraps. A desirable barrier location to particles is at the wearer/product interface because a barrier may be placed around the absorbent structure to contain not only the particles in the absorbent but also particles that may be introduced during converting that may land on the formed, wrapped absorbent structure. These unintended particles can migrate to the skin. So, there remains a need to develop materials that further reduce or eliminate superabsorbent gel particle migration that are easy and economical to manufacture.

Thus, it would be desirable to produce nonwoven fabric laminate that reduces or eliminates gel particle migration and that performs at parity of other properties of the diaper such as extensibility, fluid intake and flowback and so forth. It would also be desirable to produce a nonwoven fabric that can be used as a bodyside liner, a core wrap, a barrier tissue or as another layer between the absorbent portion of a diaper and the wearer to prevent or at least reduce gel migration from the absorbent to the wearer of the diaper.

### BRIEF DESCRIPTION OF THE INVENTION

One exemplary embodiment of the present invention provides a laminate for use in a disposable garment for the adsorption and containment of urine or other body exudates. The disposable garment includes a liquid impervious backing sheet, a nonwoven fabric laminate that includes a thin layer of fine fibers, and an absorbent material disposed between the liquid pervious bodyside liner and the liquid impervious backing sheet wherein the thin layer of fine fibers has a basis weight of less than 1.5 grams per square meter. The layer of fine fibers may consist essentially of meltblown fibers. The nonwoven fabric laminate may include at least one spunbond layer and the thin layer of fine fibers may consist essentially of a layer of meltblown fibers. In addition, the thin layer of fine fibers may be disposed between two spunbond layers. The nonwoven fabric laminate may be used as a liquid pervious bodyside liner or a layer between the absorbent material and a liquid pervious bodyside liner in a diaper. For example, the nonwoven fabric laminate can be a layer between the absorbent material and a liquid pervious bodyside liner that envelops the absorbent material. The thin layer of fine fibers may consist of a layer of fibers that has a basis weight of less than about 1 gram per square meter. Alternatively, the thin layer of fine fibers may have a basis weight of less than about 0.8 gram per square meter, less than about 0.5 gram per square meter or even less than about 0.3 gram per square meter. A nonwoven fabric laminate of the present invention may include a layer of bonded carded fibers. The spunbonded fibers of nonwoven fabric laminate of the present invention may include fibers made from a polymer selected from the group consisting of lactic acid, vinyl alcohol, and mixtures thereof.

In another embodiment, the present invention provides a nonwoven fabric laminate that consists essentially of a first layer of spunbonded fibers, a second layer of spunbonded fibers, and a layer of meltblown fibers disposed between the first layer of spunbonded fibers and the second layer of spunbonded fibers, wherein the layer of meltblown fibers has a basis weight of ranges from 0.06 grams per square meter to about 1.5 gram per square meter. The meltblown fibers may have an average diameter in the range of up to about 8 microns and the spunbonded fibers have an average diameter in the range of from about 8 microns to about 30 microns. The first spunbonded layer, the meltblown layer and the second spunbonded layer can be intermittently bonded to form the nonwoven fabric laminate. Suggested bonding techniques include thermal point bonding, ultrasonic bonding, adhesive lamination, and so forth.

### BRIEF DESCRIPTION OF THE DRAWINGS

The invention will be more fully understood and further advantages will become apparent when reference is made to various embodiments described in the following description and the accompanying drawings in which:

FIG. 1 illustrates a schematic diagram of an exemplary forming machine that can be used to form a laminate of the present invention.

FIG. 2 illustrates a cross-section view of a three-layer embodiment of a laminate of the present invention showing a three-layer configuration including an internal fine fiber barrier layer and two continuous fiber layers.

FIG. 3 illustrates a cross-section view of an alternative two-layer embodiment of a laminate of the present invention including a fine fiber barrier layer and one continuous fiber layer.

FIG. 4 illustrates a partially cut away top plan view of an exemplary personal care absorbent article, in this case a diaper, which utilizes a laminate according to the present invention.

FIG. 5 illustrates a perspective view of another absorbent article according to yet another embodiment of the present invention.

FIG. 5A illustrates a cross-sectional side view of an absorbent article according to the present invention.

FIG. 5B illustrates a cross-sectional side view of another absorbent article according to the present invention.

FIG. 5C illustrates a cross-sectional side view of another absorbent article according to the present invention.

### DETAILED DESCRIPTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

In general, the present invention is directed to laminates that include at least one fine fiber layer. In certain embodiments the laminate is a lightweight nonwoven laminate of a meltblown nonwoven material layer consisting essentially of a layer of fine fibers and a spunbonded nonwoven material layer consisting of a layer of coarser fibers. Alternatively, laminates of the present invention may include a laminate of a fine fiber, meltblown layer and another layer such as a bonded carded web of staple fibers. In certain other embodiments, the lightweight nonwoven laminate further includes a second spunbonded, continuous filament, nonwoven material layer. Furthermore, the lightweight nonwoven laminate may optionally include additional layers, for example a perforated film.

The present invention also provides disposable, absorbent garments, for example diapers, that include a liquid pervious bodyside liner that is made of or includes such a lightweight nonwoven laminate. Specifically, a laminate of the present invention can be used as the bodyside liner in a diaper or other disposable, absorbent article to contain superabsorbent gel particles or any other particles that are included in the absorbent portion of a diaper or other absorbent article. In certain desirable embodiments, the present invention provides absorbent articles that include a lightweight nonwoven laminate to contain particles that are included in the absorbent core portion of the article while allowing urine and other liquid wastes to be absorbed by the absorbent portion of the absorbent article. A laminate of the present invention may form the bodyside liner portion of the absorbent article, a wrap for the absorbent core of the absorbent article or as any other layer between the absorbent portion of the article and the wearer in order to reduce particle migration from the absorbent portion of the diaper to the interior portion of the diaper that is in contact with the skin.

Gel particles that escape from the absorbent portion of such articles have been observed to adhere to human skin especially when the particles are wet. The particles are undesirable and can be troublesome to remove. Specifically, superabsorbent gel particles have been observed on a baby's skin when the diaper is removed. The particles can be particularly difficult to remove from moist or damp skin. Laminates of the present invention provide an advantage by reducing or eliminating the amount of superabsorbent gel particles that migrate to the skin of a wearer of the absorbent article. Laminates that include a fine fiber, for example meltblown layer, are typically used as liquid barriers, for example in surgical garments to reduce blood strikethrough. These laminates have typically included a fine fiber layer of high basis weight to provide liquid barrier properties. Laminates of the present invention are fluid permeable but still retain small particles. Thus, laminates of the present invention can be used to solve the problem of migration of absorbent particles, other particles and fibers to a wearer's skin while still functioning as a liquid transfer medium.

The fine fiber layer of the present invention includes fibers having an average diameter in the range of up to about 8 microns. For applications in disposable absorbent products, the fine fiber layer includes fibers having an average diameter in the range of less than about 5 microns to even less than about 2 microns. The fine fiber layer can be formed by conventional meltblown fiber making processes. Meltblown fiber making processes are well known and are described in U.S. Pat. No. 3,849,241 to Butin et al. and U.S. Pat. No. 5,213,881 to Timmons et al., the contents of which are hereby incorporated by reference herein. For applications in disposable absorbent products, the basis weight of the fine fiber layer may be in the range of from up to about 1.5 grams per square meter (gsm) to even as low as less than about 0.06 gsm, desirably less than about 1 gsm, more desirably less than about 0.8 gsm, and even less than about 0.5 gsm. The continuous filament web has filaments with an average diameter in the range of from about 8 microns to about 30 microns. For disposable absorbent product applications, the continuous filaments have an average diameter in the range of from about 8 microns to about 25 microns. The continuous filament web can be formed by conventional spunbonded fiber making processes. Spunbonded fiber making processes are also well known and are described in for example U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. For disposable absorbent product applications, the basis weight of the continuous filament web layer may be in the range of from about 4 gsm to about 30 gsm or in the range of from about 10 gsm to about 20 gsm. The fine fiber layer and the continuous fiber layer can bonded intermittently for a total basis weight not to exceed about 55 gsm and the amount of fine fibers in the laminate based on the weight of the laminate can be as low as 10 weight percent, 5 weight percent and even as low as 1 weight percent. Advantageously for disposable absorbent product applications the laminate basis weight in accordance with the invention is extremely low and within the range of up to about 10 gsm and the fine fibers constitute a low proportion of the laminate in the range of about 5 percent to about 25 percent by weight. When desired as a liner, laminates of the present invention can have improved fluid permeability as measured by hydrostatic head of less than 15 cm and breathability as measured in terms of Frazier porosity of at least 50 scfm.

Desirable commercial embodiments include spunbond continuous filament web and meltblown fine fiber webs as the respective layers. The meltblown fine fiber nonwoven web layer can be formed by a melt-blown web forming process such as the process described in coassigned U.S. Pat. No. 5,213,881 to Timmons et al. or U.S. Pat. No. 5,492,751 to Butt, Sr. et al. The present invention can be carried out with thermoplastic resins, for example polyolefins including predominantly propylene polymer but which may include, polyethylene, or other alphaolefins polymerized with Ziegler-Natta catalyst technology, and copolymers, terpolymers, or blends thereof. Polypropylene resins are desirable for the continuous filament web layer. However, the continuous filaments can be made from inherently wettable, nonpolyolefin resins such as polymers and copolymers of vinyl acetate or lactic acid. Alternatively, the filaments or a nonwoven web of the filaments can be treated with one or more surfactants to improve the wettability of the fibers and the resulting nonwoven web.

The meltblown fine fibers and fine fiber nonwoven webs can be formed from a propylene polymer resin having a broad molecular weight distribution and having a high melt flow rate which resin is modified by the addition of a small amount of peroxide prodegradant or heating prior to processing to achieve an even higher melt flow rate (lower viscosity). For example, the propylene resin can be modified using organic peroxides. Examples of modifying polypropylene using organic peroxides is described in U.S. Pat. No. 4,451,589 which is hereby incorporated by reference herein. In general, the present invention may start with a propylene polymer in the form of reactor granules which polymer has a molecular weight distribution of 3.6 to 4.8 M_{w}/Mₙ, advantageously 3.6 to 4.0 M_{w}/Mₙ and a melt flow rate of about 400 gms/10 min to 3000 gms/10 min at 230° C. Such a molecular weight reactor granule polymer is then modified to reduce and narrow the polymer's molecular weight distribution to a range from 2.2 to 3.5 M_{w}/Mₙ by the addition of up to 3000 parts per million (ppm) of peroxide prodegradant. During the meltblowing process, the modified reactor granule polymer increases in melt flow rate from 400 gms/10 min. to 3000, for example, to a range between 800 up to 5000 gms/10 min at 230° C. Particularly advantageous embodiments for disposable aborbent applications include a polypropylene resin in the form of a reactor granule having a starting molecular weight distribution of 3.6 to 4.8 M_{w}/Mₙ and a melt flow rate of from 600 to 3000 gms/10 min. at 230° C. which is combined with a small amount of peroxide prodegradant, less than 500 ppm, to produce a modified polypropylene having a very high melt flow rate of up to 5000 gms/10 min. at 230° C. and a narrower molecular weight distribution of 2.8 to 3.5 M_{w}/Mₙ.

Alternatively, an improved fine fiber web for use as a barrier layer can be formed by utilizing a resin, particularly polypropylene, having a narrow molecular weight distribution and having a lower melt flow rate which resin is modified by the addition of a larger amount of peroxide prodegradant prior to melt-blowing to achieve a high melt flow rate. The starting reactor granule polypropylene resin in this case has a molecular weight distribution between 4.0 and 4.8 M_{w}/Mₙ and a melt flow rate ranging from 400 to 1000 gms/10 min. at 230° C. The polypropylene resin is modified by adding peroxide in amounts ranging from 500 to 3000 ppm (the higher amounts of peroxide being used in connection with the lower initial melt flow rate). The modified polypropylene resin has a melt flow rate, up to about 3000 gms/10 min. at 230° C. and a narrow molecular weight distribution of 2.2 to 2.8 M_{w}/Mₙ, for example.

As a specific example, the starting polypropylene resin for the fine fiber web of the lightweight nonwoven laminate of the present invention may be a polypropylene reactor granule which resin has a molecular weight distribution between 3.6 and 4.8 M_{w}/Mₙ, has a melt flow rate of up to 3000 gms/10 min. at 230° C., and is treated with about 500 ppm of peroxide to produce a modified resin having a melt flow rate greater than 2000 gms/10 min. at 230° C. and a molecular weight distribution of from 2.8 to 3.5 M_{w}/Mₙ.

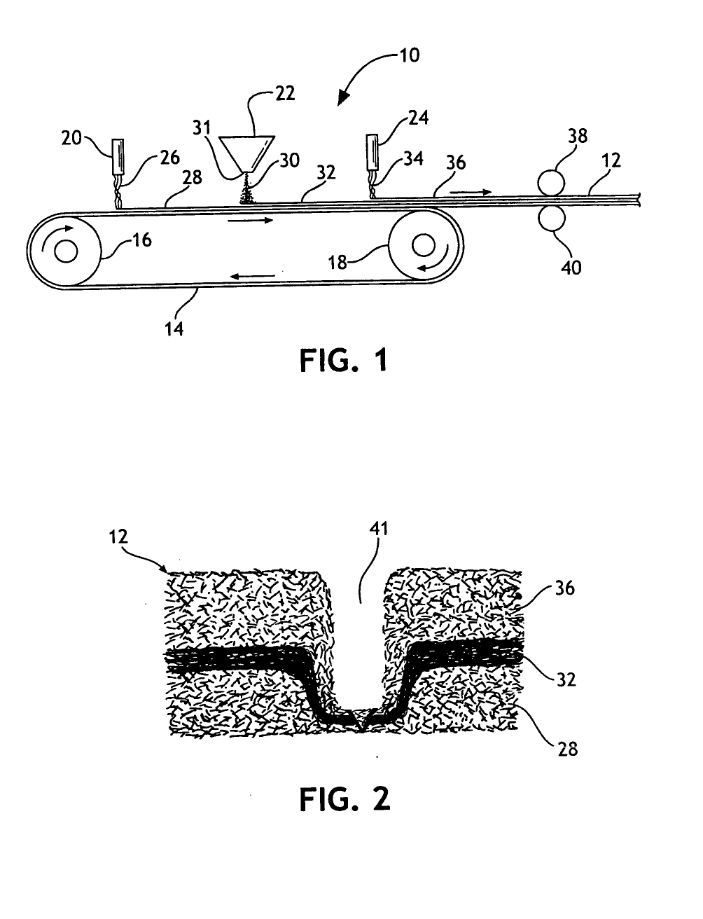

Turning to FIG. 1, there is shown schematically a forming machine 10 which may be used to produce a nonwoven fabric laminate 12 having a fine fiber meltblown layer 32 and outer continuous filaments spunbond layer 28 in accordance with the present invention. Particularly, the forming machine 10 includes of an endless foraminous forming belt 14 wrapped around rollers 16 and 18 so that the belt 14 is driven in the direction shown by the arrows. The illustrated forming machine 10 has three stations: spunbond station 20, meltblown station 22, and spunbond station 24 to produce a spunbond/meltblown/spunbond (SMS) laminate as described in U.S. Pat. No. 4,041,203 which is hereby incorporated by reference herein. It should be understood that more than three forming stations may be utilized to build up additional layers to produce a laminate of more layers or having a higher basis weight and each forming station can have multiple banks or dies. Alternatively, each of the laminate layers may be formed separately, rolled, and later converted to the fabric laminate off-line. In addition the fabric laminate 12 could be formed of more than or less than three layers depending on the requirements for the particular end use for the fabric laminate 12. For example, for some applications it may be preferred to have a laminate of one fine fiber meltblown web layer and one spunbond continuous filament web layer. Particularly, for extremely lightweight applications and/or high liquid permeability applications a two-layer laminate is desirable.

The spunbond stations 20 and 24 can be conventional extruders with spinnerets which form continuous filaments of a polymer and deposit those filaments onto the forming belt 14 in a random interlaced fashion. The spunbond stations 20 and 24 may include one or more spinnerets heads depending on the speed of the process and the particular polymer or polymers being used. Forming spunbonded material is conventional in the art, and the design of such a spunbonded forming station is within the ability of those of ordinary skill in the art. The nonwoven spunbonded webs 28 and 36 can be formed using known methods such as those described and illustrated in the following patents: U.S. Pat. No. 3,692,618 to Dorschner et al.; U.S. Pat. Nos. 3,338,992 and 3,341,394 to Kinney; U.S. Pat. No. 3,502,538 to Levy; U.S. Pat. Nos. 3,502,763 and 3,909,009 to Hartmann; U.S. Pat. No. 3,542,615 to Dobo et al.; Canadian Patent no. 803,714 to Harmon; U.S. Pat. No. 3,802,817 to Matsuki et al. and U.S. Pat. No. 4,340,563 to Appel et al. Other methods for forming a nonwoven web having continuous filaments of a polymer are contemplated for use with the present invention.

Spunbonded materials prepared with continuous filaments generally have at least three common features. First, the polymer is continuously extruded through a spinneret to form discrete filaments. Thereafter, the filaments are drawn either mechanically or pneumatically without breaking in order to molecularly orient the polymer filaments and achieve increased tenacity. Lastly, the continuous filaments are deposited in a substantially random manner onto a carrier belt to form a web and are then bonded to form a laminate. Particularly, the spunbond station 20 produces spunbond filaments 26 from a fiber forming polymer. The filaments are randomly laid on the belt 14 to form a spunbonded external layer 28. The spunbonded layer can be optionally bonded. The fiber forming polymer is described in greater detail below.

The meltblown station 22 consists of a die 31 which is used to form microfibers 30 having an average diameter in the range of up to about 8 microns or even as low as an average diameter in the range of about 5 microns, 2 microns and even as low as 1.5 microns. Meltblown station 22 may consist of multiple meltblown die tips (not shown). For example, meltblown station 22 may include 2, 3, 4 or more die tips. The throughput of the die 31 is specified in pounds of polymer melt per inch of die width per hour (PIH). As the thermoplastic polymer exits the die 31, high pressure fluid, usually heated air, attenuates and spreads the polymer stream to form microfibers 30. The microfibers 30 are randomly deposited on top of the spunbond layer 28 and form a meltblown layer 32. The distance between the die tip and the forming wire is typically between about 3 and 18 inches. The short distance and the uniform spacing of holes in the die tip produce a very uniform fiber laydown that is desired for particle filtration performance. The construction and operation of the meltblown station 22 for forming microfibers 30 and meltblown layer 32 is considered conventional, and the design and operation are well within the ability of those of ordinary skill in the art. Such skill is demonstrated by NRL Report 4364, “Manufacture of Super-Fine Organic Fibers”, by V. A. Wendt, E. L. Boon, and C. D. Fluharty; NRL Report 5265, “An Improved Device for the Formation of Super-Fine Thermoplastic Fibers”, by K. D. Lawrence, R. T. Lukas, and J. A. Young; and U.S. Pat. No. 3,849,241, issued Nov. 19, 1974, to Buntin et al. Still other methods for forming a nonwoven web of microfibers are known and are contemplated for use with the present invention.

The meltblown station 22 produces fine fibers 30 from a fiber forming polymer which will be described in greater detail below. The fibers 30 are randomly deposited on top of spunbond layer 28 to form a meltblown layer 32. Meltblown layer 32 may be an internal fine fiber layer in a SMS laminate. For liner and core wrap applications, the meltblown barrier layer 32 may have a basis weight of less than about 2 gsm, more desirably less than about 1 gsm, even more desirably less than about 0.8 gsm, still even more desirably less than about 0.5 gsm and even as low as more desirably less than about 0.3 gsm. For core wrap applications, the meltblown barrier layer 32 may have a basis weight of less than about 14 gsm, desirably less than 2 gsm, more desirably less than about 1 gsm, even more desirably less than about 0.8 gsm, still even more desirably less than about 0.5 gsm and even as low as more desirably less than about 0.3 gsm.

After the internal layer 32 has been deposited by the meltblown station 22 onto layer 28, spunbond station 24 produces spunbond filaments 34 which are deposited in random orientation on top of the melt-blown layer 32 to produce external spunbond layer 36. For applications, for example, the layers 28 and 36 each have a basis weight of commonly from about 3 gsm to about 30 gsm, more advantageously about 5 gsm to about 20 gsm. The polymer that is used to make the spunbond layers and the basis weight of the spunbond layers 28 and 36 may be the same or different. The resulting SMS fabric laminate web 12, shown in greater detail in FIG. 2, is then fed through bonding rolls 38 and 40. The surfaces of one or both of the bonding rolls 38 and 40 are provided with a raised pattern such as spots or grids. The bonding rolls are heated to the softening temperature of the polymer used to bond the layers of the web 12. As the web 12 passes between the heated bonding rolls 38 and 40, the material is compressed and heated by the bonding rolls in accordance with the pattern on the rolls to create a pattern of discrete areas, such as 41 shown in FIG. 2, which areas are bonded from layer to layer and are bonded with respect to the particular filaments and/or fibers within each layer. Such discrete area or spot bonding is well-known in the art and can be carried out as described by means of heated rolls or by means of ultrasonic heating of the web 12 to produced discrete area thermally bonded filaments, fibers, and layers. In accordance with conventional practice described in U.S. Pat. No. 4,041,203 to Brock et al., it is desirable for the fibers of the meltblown layer in the fabric laminate to fuse within the bond areas while the filaments of the spunbonded layers retain their integrity in order to achieve good strength characteristics. For heavier basis weight laminates, for example, sonic bonding as described in U.S. Pat. No. 4,374,888, incorporated herein by reference, is desired. The laminate can be bonded using other bonding methods such as point bonding and by using various bonding patterns such as a wire weave pattern. Point bonding and bonding patterns are described in U.S. Pat. No. 5,599,420 to Yeo et al. which is hereby incorporated be reference herein.

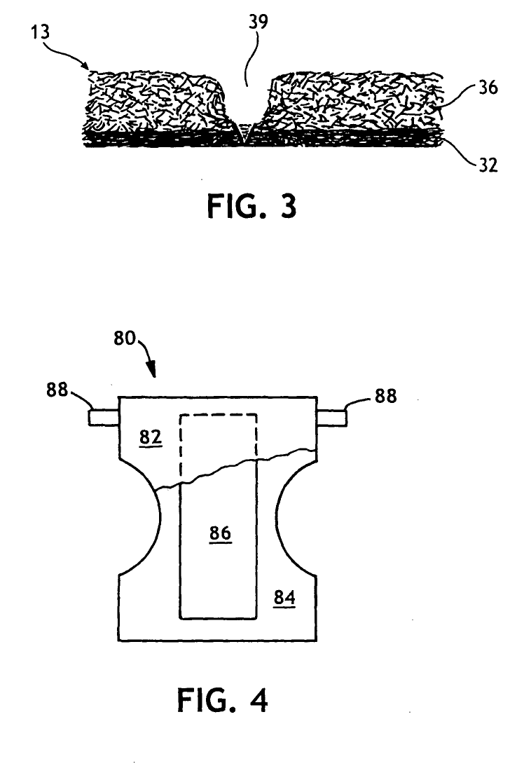

At least one alternative embodiment, more precisely one group of alternative embodiments, is illustrated in FIG. 3. FIG. 3 is a cross-section similar to FIG. 2 showing a two layer laminate 13 of the present invention which includes one fine fiber layer 32 and one continuous filament layer 36 combined by thermal bond 39.

In accordance with the invention, the total basis weight of the laminate is in the range generally of up to about 55 gsm, more desirably less than about 34 gsm for applications such as liners and core wraps for absorbent product applications, still more desirably less than about 20 gsm and even less than 10 gsm for liner and core wrap applications. The amount of fine fibers compared to continuous filaments is generally at least about 1 percent (by weight) generally and up to about 30 weight percent based on total weight of fine fiber layer and the continuous filament layer(s). Laminates of the present invention include, but are not limited to: SMSMS, SSMS, SMMMS, and other possible multiple layer combinations of spunbond and meltblown banks.

A nonwoven fabric laminate of the present invention, for example the three-layer laminate 12 illustrated and described with reference to FIG. 2, a two-layer laminate 13 illustrated and described with reference to FIG. 3 or a laminate of additional layers, may be used in a wide variety of applications, not the least of which includes personal care absorbent articles such as diapers, training pants, incontinence devices and feminine hygiene products such as sanitary napkins. An exemplary article 80, in this case a diaper, is shown generally in FIG. 4 of the drawings. Other more complicated diaper constructions are known and are described and illustrated in greater detail in for example U.S. Pat. No. 5,520,673 to Yarbrough et al. and U.S. Pat. No. 6,217,890 to Paul et al., both of which are herby incorporated be reference herein. Referring to FIG. 4 of the present invention, most such personal care absorbent articles 80 include a liquid permeable top sheet or liner 82, a back sheet or outercover 84 and an absorbent core 86 disposed between and contained by the top sheet 82 and back sheet 84. Articles 80 such as diapers may also include some type of fastening means 88 such as adhesive fastening tapes or mechanical hook and loop type fasteners.

Specific examples of disposable diapers suitable for use in the present invention, and other components suitable for use therein, are disclosed in the following U.S. patents and U.S. patent applications: U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmer et al.; U.S. Pat. No. 5,192,606 issued Mar. 9, 1993, to Proxmire et al.; U.S. Pat. No. 5,415,644 issued May 16, 1995, to Enloe; and U.S. Pat. No. 5,509,915 all of which are hereby incorporated herein by reference. Other suitable components include, for example, containment flaps and waist flaps.

A nonwoven fabric laminate of the present invention by itself, or in other forms, such as a component in multilayer laminate including additional layers or some other composite structure, may be used to form various portions of the article including, but not limited to, the top sheet 82, as a wrap for the absorbent core 86 or a layer between the absorbent core 86 and the interior of the absorbent article 80, that Is as a layer between the absorbent core 86 and a wearer of the article. In one example a laminate of the present invention is a wrap for the absorbent core 86 and/or the liner 82 portion of the diaper and can be formed completely from or include one of the laminates described herein to minimize the migration of particles from the absorbent core to the wearer's skin. If a laminate of the present invention is to be used as a top sheet 82, a core wrap material or as a layer between the absorbent core and a wearer, the laminate is desirably liquid permeable while retaining absorbent particles that may be contained in the absorbent core 86. Absorbent particles may have diameters as small as 0.001 inches, therefore it would be desirable that the fine fiber layer of the laminate has holes no larger than 0.001 inches in diameter. For example, a theoretically, perfect laid down grid of one micron polypropylene fibers would act as a barrier for 0.001 inch particles at a basis weight of 0.06 gsm. Thus, laminates of the present invention may include a fine fiber or meltblown layer having a basis weight of at least 0.06 grams per square meter (gsm). Laminates of the present invention with their fine fiber layers and resulting small pore size distribution can have superior particle retention and water permeability properties.

As previously stated, laminates of the present invention can be used as a wrap for the absorbent core of an absorbent article or as a layer between the absorbent core and the interior of the absorbent article. For example, laminates of the present invention may be substituted as the core wrap material in a diaper such as the core wrap material described in U.S. Pat. No. 5,458,592 which is hereby incorporated be reference herein. As such, laminate materials of the present invention are particularly well-suited for containing absorbent cores which are made partially or completely from particulate matter such as superabsorbent particles. The laminate of the present invention is particularly useful for reducing the migration of superabsorbent particles in articles that contain superabsorbent particles. For example, the laminate is particularly useful in articles having an absorbent portion that has a high superabsorbent particle content such as greater than 50 weight percent. It should be understood, however, that the present invention is not restricted to use with superabsorbent particles but any particulate material such as odor absorbing and ion exchange resin particles and controlled release agents such as moisturizers, emollients and perfumes which require retention.

A “superabsorbent” or “superabsorbent material” refers to a water-swellable organic or inorganic material capable, under the most favorable conditions, of absorbing at least about 20 times its weight and, more desirably, at least about 30 times its weight in an aqueous solution containing 0.9 weight percent sodium chloride. Organic materials suitable for use as a superabsorbent material in conjunction with the present invention can include natural materials such as agar, pectin, guar gum, and so forth; as well as synthetic materials, such as synthetic hydrogel polymers. Such hydrogel polymers include, for example, alkali metal salts of polyacrylic acids, polyacrylamides, polyvinyl alcohol, ethylene maleic anhydride copolymers, polyvinyl ethers, methyl cellulose, carboxymethyl cellulose, hydroxypropylcellulose, polyvinylmorpholinone; and polymers and copolymers of vinyl sulfonic acid, polyacrylates, polyacrylamides, polyvinylpyrridine, and so forth. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking may, for example, be accomplished by irradiation or by covalent, ionic, van der Waals, or hydrogen bonding. The superabsorbent materials may be in any form suitable for use in absorbent composites including particles, fibers, flakes, spheres, and so forth. Such superabsorbents are usually available in particle sizes ranging from about 20 to about 1000 microns. The absorbent core 86 can contain from 0 to 100 percent superabsorbent by weight based upon the total weight of the absorbent core. Typically an absorbent core 86 for a personal care absorbent product will include superabsorbent particles and, optionally, additional absorbent material such as absorbent fibers including, but not limited to, wood pulp fluff fibers, synthetic wood pulp fibers, synthetic fibers and combinations of the foregoing. Wood pulp fluff such as CR-1654 wood pulp available from Bowater Incorporated of Greenville, S.C. is an effective absorbent supplement. A common problem with wood pulp fluff, however, is its lack of integrity and its tendency to collapse when wet. As a result, it is often advantageous to add a stiffer reinforcing fiber into the absorbent core such as polyolefin meltblown fibers or shorter length staple fibers. Such combinations of fibers are sometimes referred to as “coform”. The manufacture of meltblown fibers and combinations of meltblown fibers with superabsorbents and/or wood pulp fibers are well known. Again, meltblown webs are made from fibers formed by extruding a molten thermoplastic material through a plurality of fine, usually circular dye capillaries as molten threads or filaments into a high-velocity heated air stream which attenuates the filaments of molten thermoplastic material to reduce their diameters. Shaped and/or multicomponent fibers may also be used. Thereafter, the meltblown fibers are carried by the high-velocity gas stream and are deposited on a collecting surface to form a web of randomly dispersed meltblown fibers. The meltblown process is well known and is described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. No. 3,849,241, issued Nov. 19, 1974 to Buntin et al. To form “coform” materials, additional components are mixed with the meltblown fibers as the fibers are deposited onto a forming surface. For example, superabsorbent particles and/or staple fibers and/or wood pulp fibers may be injected into the meltblown fiber stream so as to be entrapped and/or bonded to the meltblown fibers. See, for example, U.S. Pat. No. 4,100,324 to Anderson et al.; U.S. Pat. No. 4,587,154 to Hotchkiss et al., U.S. Pat. Nos. 4,604,313; 4,655,757 and 4,724,114 to McFarland et al. and U.K. Patent GB 2,151,272 to Minto et al., all of which are incorporated herein by reference in their entirety.

Laminates of the present invention that are intended to be used as a core wrap material or as a bodyside liner can be designed and formed to include a fibrous nonwoven web layer made from fine diameter thermoplastic fibers with particular pore sizes and air permeability. By thermoplastic fibers it is meant fibers which are formed from polymers such that the fibers can be bonded to themselves using heat or heat and pressure. While not being limited to the specific method of manufacture, meltblown fibrous nonwoven webs have been found to work particularly well. With respect to polymer selection, polyolefin fibers and especially polypropylene-based polymers have been found to work well. The general manufacture of such meltblown fibrous nonwoven webs is well known. See for example, the previously mentioned meltblown patents referred to above. The fibers may be hydrophilic or hydrophobic, though it is desirable that the resultant web, bodyside liner and core wrap be hydrophilic. As a result, the fibers may be formed from inherently wettable resins, such as polylactic acid, polyvinyl alcohol resins or polyesters, or can be treated to be hydrophilic as by the use of a surfactant treatment.

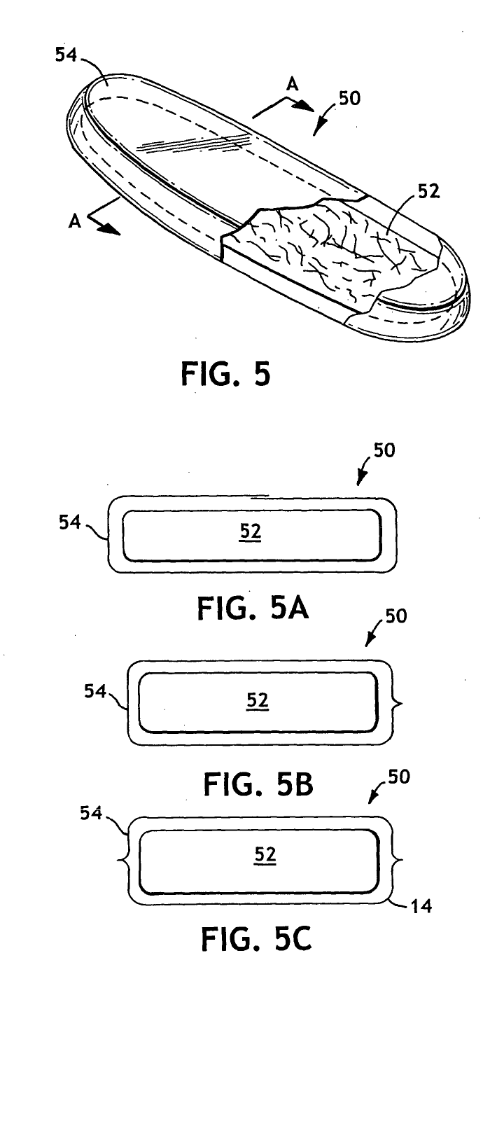

In order to function well as a core wrap, the meltblown web should have certain specific properties. A common problem with paper tissue wrap is that it has inadequate strength in the wet state. Typically a paper tissue wrap will have a wet to dry strength ratio in either the machine direction (MD) or cross-machine direction (CD) as measured by the test method outlined below of less than 0.5. In contrast, a absorbent core wrap of the present invention, illustrated as layer 54 in FIGS. 5, 5A, 5B and 5C, should have wet to dry strength ratios above 0.5 and sometimes 1.0 or higher. FIG. 5 illustrates a an absorbent core 52 that is wrapped or otherwise enveloped by a laminate of the present invention to form a wrapped absorbent core 50 that can be used as an absorbent portion of a diaper, for example, that does not release absorbent particles on the wearer.

The laminate wrap material 54 may be simply folded around the absorbent core 52 as illustrated in cross-sectional FIG. 5A, wrapped and bonded around one edge, as illustrated in FIG. 5B, two layers bonded around the absorbent core 52 as illustrated in FIG. 5C or unsealed. Alternatively, the laminate may be included as an additional layer in between the absorbent portion of an absorbent article and the skin contacting or body sideliner of the absorbent article. When using a two-layer nonwoven laminate such as that illustrated in FIG. 3 as the top sheet 82, it is may be advantageous to orient the fine fiber layer of laminate facing the absorbent core of the diaper to protect the fine fiber layer from damage and preserve the layers particle barrier properties. Although the present invention is illustrated by application to a diaper, laminates of the present invention can be used in other absorbent articles including, but not limited to, incontinence garments, pantiliners and so forth. Other uses for the multilayer nonwoven laminates according to the present invention may include, but are not limited to, surgical drapes, gowns, wipers, barrier materials, other garments and articles of clothing or portions thereof including such items as workwear and lab coats, and filtration materials such as water filters and so forth.

In one embodiment, a laminate of the present invention is surface treated with one or more surfactants to improve the wettability of the laminate. One commercially available surfactant that can be used to surface treat a laminate of the present invention can be obtained from Union Carbide Chemicals and Plastics Company, Inc., of Danbury, Conn., U.S.A. under the trade designation TRITON X-102. The fabric laminate may be surface treated with about 0.3 weight percent of a surfactant mixture that contains a mixture of AHCOVEL Base N-62 and GLUCOPON 220 UP surfactant in a 3:1 ratio based on a total weight of the surfactant mixture. AHCOVEL Base N-62 can be obtained from Uniqema Inc., a business having offices in New Castle, Del., and includes a blend of hydrogenated ethoxylated castor oil and sorbitan monooleate. GLUCOPON 220 UP can be obtained from Cognis Corporation, a business having offices in Ambler, Pa., and includes alkyl polyglycoside. The surfactant may be applied by any conventional means, such as dip and squeeze, spraying, printing, brush coating or the like. The surfactant may be applied to the entire laminate or may be selectively applied to particular sections of the laminate, such as the medial section along the longitudinal centerline of a diaper or other personal care product, to provide greater wettability of such sections. Exemplary surface treatment compositions and methods of applying surface treatment compositions are described in U.S. Pat. Nos. 5,057,361; 5,683,610 and 6,028,016 which also are hereby incorporated by reference herein.

Run-Off Test Procedure

All run-off data reported herein were obtained using the following run off test. Once a fibrous porous web material is selected for testing, a sheet of the material measuring 325 mm long, in the machine direction, and 150 mm wide is laid against the absorbent of a HUGGIES® brand diaper size 3. The test material is smoothed out on the absorbent by rubbing it lengthwise 3 times with a downward hand motion. The test material functions as a core wrap or a liner and particle barrier between the absorbent and anything above the absorbent, for example a baby's bottom. The absorbent was obtained from a diaper which had its inner body side liner and surge layer removed. Note that in some cases a surge layer that had been removed from the diaper was laid on top of the absorbent before the liner was applied. Any elastic leg, flap, and elastic waist material was also removed so that the absorbent will assume a generally flat configuration. The polyethylene film backing and tissue top cover are left on the absorbent to give it integrity so that it can be handled. The absorbent is cut to about 100 mm wide and 300 mm long. The 300 mm is measured from the front, top end of the diaper absorbent and the excess cut off. Prior to placement of the test material on top of the absorbent, the absorbent is placed, film side down, on a water impervious plane inclined at 30 degrees to horizontal and terminating, at its lower edge, with a V-shaped raised edge which directs any fluid impacting the raised edge toward a hole in the plane which is centrally located at the vertex of the “V”. A 250 milliliter beaker is located beneath the hole to collect any fluid passing therethrough. The absorbent is positioned on the inclined plane so that the top of the front panel (the uncut end) constitutes its upper free edge on the inclined plane. The lower free edge is located approximately 200 millimeters from the point of impact of testing fluid. A funnel arrangement with a stopcock is positioned above the diaper approximately 200 millimeters from the lower free edge of the absorbent and in such manner that an approximate 50 millimeter clearance exists between the top of the absorbent and the lower tip of the funnel. The lower free edge of the absorbent is located approximately 50 millimeters up the incline from the hole in the inclined plane. The test material is positioned over the absorbent so that it overhangs it by about 25 millimeters. Approximately one hundred milliliters of room temperature tap water is poured into the funnel with the stopcock closed. The funnel stopcock is opened to allow the test fluid to flow from the funnel onto the test material at a rate so that all 100 milliliters is dispensed in about 18+/−2 seconds. Fluid which is collected within the 250 milliliter beaker is “run-off”. The amount of run-off for a given test is measured in grams.

Superabsorbent Material (SAM) Shake Test Procedure

The SAM Shake Test was preformed as follows:

1. Measure 25 g of SAM per sample.

2. Affix web of material to span across the top of a U.S. Standard 8 inch diameter, 2 inch height, #20 Mesh sieve (#20 retains particles>850 microns in diameter).

3. Place a second U.S. Standard #20 Mesh sieve on top of the web of material so that the sample material is taut and secure between the sieves.

4. Distribute the 25 g of SAM in the top sieve.
The 25 grams of SAM particles consisted of:
   99.7% by weight of particles<850 microns,
   72.6% by weight of particles<600 microns,
   23.8% by weight of particles<300 microns,
   1.0% by weight of particles<90 microns, and
   0.3% by weight of particles<45 microns.

5. Place the top sieve, sample material, bottom sieve, and a catch pan into Ro-Tap Sieve Shaker (W. S. Tyler, Inc part # RX29) instrument and set timer for 10 minutes.

6. After ten minutes remove the two sieves and material sample from the catch pan.

7. Weigh the amount of SAM that was not retained by the material sample that has been collected in the catch pan.

8. The % SAM retained is calculated by subtracting the amount of SAM in grams in the catch pan from the original 25 grams of SAM introduced into the top sieve and then dividing the result by 25 grams of SAM.

### EXAMPLE 1

A SMS fabric laminate (80 percent SB and 20 percent MB) was produced by forming and laminating a first 0.14 (4.75 gsm) osy spunbond layer, a 0.07 osy (2.4 gsm) meltblown layer, and a second 0.14 osy spunbond layer at a line speed of about 1996 feet per minute (fpm). The SMS laminate having an overall basis weight 0.35 osy (1.9 gsm) was and was heated online with a Hot Air Knife (HAK) at 435° F. as described in U.S. Pat. No. 6,019,152 and then thermally bonded using a wire weave bond pattern. The SMS laminate was surface treated off-line with an aqueous treatment solution consisting of water and about 0.3 weight percent of a surfactant mixture that consisted of a mixture of AHCOVEL Base N-62 and GLUCOPON 220 UP surfactant at a 3:1 ratio based on a total weight of the surfactant mixture using the dip and squeeze method and targeting an 80 percent wet pick-up value.

The 0.35 osy laminate was tested using the SAM shake test. The 0.35 osy SMS laminate achieved a 99.6 percent SAM retention level using the SAM shake test. Typically, a control example of a conventional spunbond liners at 0.6 osy achieved about 83 percent SAM retention level using the SAM shake test. Thus, the 0.35 osy SMS laminate of Example 1 provides superior particle retention properties. The 0.35 osy SMS laminate of Example 1 was also tested for Run-Off using the above-described procedure measured 1.9 grams of run-off without the surge layer and 1.5 grams of run-off with the surge layer. The control example of a conventional necked spunbond liners of about 0.6 osy measured 7.9 grams of run-off without the surge layer and 17.9 grams of run-off with the surge layer. A run-off of less than 50 grams is acceptable and a run-off of less than 20 grams is desired.

### EXAMPLE 2

A SMS fabric laminate (80 percent SB and 20 percent MB) was produced by forming and laminating a first 0.18 osy (6.1 gsm) spunbond layer, a 0.09 osy (3.0 gsm) meltblown layer, and a second 0.18 osy spunbond layer at a line speed of about 1843 fpm. The necked SMS laminate had an overall basis weight 0.45 osy (15.3 gsm). This SMS laminate was also surface treated with an aqueous treatment solution consisting of water and about 0.3 weight percent of a surfactant mixture that consisted of a mixture of AHCOVEL Base N-62 and GLUCOPON 220 UP surfactant at a 3:1 ratio based on a total weight of the surfactant mixture using the dip and squeeze method and targeting an 80 percent wet pick-up value.

The 0.45 osy laminate was tested using the SAM shake test. The 0.45 osy SMS laminate of Example 2 achieved a 100 percent SAM retention level using the SAM shake test. The 0.45 osy SMS laminate of Example 2 was also tested for Run-Off using the above-described procedure and measured 2.3 grams of run-off without the surge layer and 4.9 grams of run-off with the surge layer.

### EXAMPLE 3

A SMS fabric laminate (90 percent SB and 10 percent MB) was produced by forming and laminating a first 0.1575 osy (5.3 gsm) spunbond layer, a 0.035 osy (1.2 gsm) meltblown layer, and a second 0.1575 osy spunbond layer at a line speed of about 1996 fpm. The necked SMS laminate had an overall basis weight 0.35 osy (11.9 gsm). This SMS laminate of Example 3 was also surface treated with the same treatment solution using the dip and squeeze method at a targeting 80 percent wet pick-up.

The 0.35 osy laminate was tested using the SAM shake test. The 0.35 osy SMS laminate of Example 3 achieved a 98.8 percent SAM retention level using the SAM shake test. The 0.35 osy SMS laminate of Example 3 was also tested for Run-Off using the above-described procedure and measured 0.9 grams of run-off without the surge layer and 0.7 grams of run-off with the surge layer.

### EXAMPLE 4

A SMS fabric laminate (90 percent SB and 10 percent MB) was produced by forming and laminating a first 0.2025 osy (6.9 gsm) spunbond layer, a 0.045 osy (1.5 gsm) meltblown layer, and a second 0.2025 osy spunbond layer at a line speed of about 1552 fpm. The necked SMS laminate had an overall basis weight 0.45 osy 15.3 gsm). This SMS laminate of Example 4 was also surface treated with the same treatment solution using the dip and squeeze method at a targeting 80 percent wet pick-up.

The 0.45 osy SMS laminate of Example 4 achieved a 99.6 percent SAM retention level using the SAM shake test. The 0.45 osy SMS laminate of Example 4 was also tested for Run-Off using the above-described procedure and measured 1.0 grams of run-off without the surge layer and 1.3 grams of run-off with the surge layer.

### EXAMPLE 5

A necked SMS fabric laminate with a wire weave bond pattern was produced having a first 0.3375 osy (11.4 gsm) spunbond layer, a 0.072 osy (2.4 gsm) meltblown layer, and a second 0.3375 osy spunbond layer. The necked SMS laminate had an overall basis weight 0.72 osy and was necked at 27 percent, i.e. to 83% of its unnecked width. The SMS laminate was surface treated with an aqueous foam treatment solution consisting of water and about 20 weight percent of a surfactant mixture that consisted of a mixture of AHCOVEL Base N-62 and GLUCOPON 220 UP surfactant at a 3:1 ratio based on a total weight of the surfactant mixture using the dip and squeeze method and targeting an 80 percent wet pick-up value.

The 0.72 osy laminate was tested using the SAM shake test. The 0.72 osy SMS laminate achieved a 99.6 percent SAM retention level using the SAM shake test. In contrast, current necked spunbond liners achieve about 80 percent SAM retention level using the SAM shake test. Thus, the 0.72 osy SMS laminate of Example 5 provides superior particle retention properties. Additionally, the 0.72 osy SMS laminate of Example 5 was tested for CD extensibility using a Sintec Tensile Tester using a 3 inch wide sample and a jaw separation of 3 inches. The 0.72 osy SMS laminate of Example 5 had 40 percent extension at 79 grams.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

1. A disposable garment for the adsorption and containment of urine or other body exudates, the disposable garment comprising:
a. a liquid impervious backing sheet;
b. a liquid pervious, nonwoven fabric laminate that comprises a thin layer of fine fibers having an average diameter of up to about 8 microns; and
c. an absorbent material disposed between the liquid pervious nonwoven fabric laminate and the liquid impervious backing sheet
wherein the thin layer of fine fibers has a basis weight of less than 1.5 grams per square meter.

2. The disposable garment of claim 1 wherein the layer of fine fibers consist essentially of meltblown fibers.

3. The disposable garment of claim 2 wherein the nonwoven fabric laminate comprises at least one spunbond layer and the thin layer of fine fibers consists essentially of a layer of meltblown fibers.

4. The disposable garment of claim 3 wherein the thin layer of fine fibers consists essentially of a layer of meltblown fibers and the nonwoven fabric laminate consists essentially of the thin layer of fine fibers disposed between two spunbond layers.

5. The disposable garment of claim 1 wherein the nonwoven fabric laminate is a liquid pervious bodyside liner or a layer between the absorbent material and a liquid pervious bodyside liner.

6. The disposable garment of claim 1 wherein the nonwoven fabric laminate is a liquid pervious bodyside liner.

7. The disposable garment of claim 1 wherein the nonwoven fabric laminate is a layer between the absorbent material and a liquid pervious bodyside liner.

8. The disposable garment of claim 3 wherein the absorbent material comprises particles selected from the groups consisting of surperabsorbent particles, synthetic polymer particles, carbon particles and combinations thereof

9. The disposable garment of claim 1 wherein the thin layer of fine fibers consists of a layer of fibers that has a basis weight of less than about 1 gram per square meter.

10. The disposable garment of claim 1 the thin layer of fine fibers consists of a layer of fibers that has a basis weight of less than about 0.8 gram per square meter.

11. The disposable garment of claim 1 the thin layer of fine fibers consists of a layer of fibers that has a basis weight of less than about 0.5 gram per square meter.

12. The disposable garment of claim 1 the thin layer of fine fibers consists of a layer of fibers that has a basis weight of less than about 0.3 gram per square meter.

13. The disposable garment of claim 1 further comprising particles of a superabsorbent material dispersed in the absorbent material.

14. The disposable garment of claim 1 wherein the nonwoven fabric laminate further comprises a layer of bonded carded fibers.

15. The disposable garment of claim 1 wherein the spunbonded fibers comprise fibers made from a polymer selected from the group consisting of lactic acid, vinyl alcohol, and mixtures thereof.

16. A nonwoven fabric laminate consisting essentially of:
a. a first layer of spunbonded fibers,
b. a second layer of spunbonded fibers,
c. a layer of meltblown fibers disposed between the first layer of spunbonded fibers and the second layer of spunbonded fibers,
wherein the layer of meltblown fibers has a basis weight of ranges from 0.06 grams per square meter to about 1 gram per square meter.

17. The nonwoven fabric laminate of claim 16 wherein the basis weight of the layer of meltblown fibers is less than about 0.8 grams per square meter.

18. The nonwoven fabric laminate of claim 16 wherein the basis weight of the layer of meltblown fibers is less than about 0.5 grams per square meter.

19. The nonwoven fabric laminate of claim 16 wherein the basis weight of the layer of meltblown fibers is less than about 0.3 grams per square meter.

20. The nonwoven fabric laminate of claim 16 wherein the layer of meltblown fibers consists of fibers having an average diameter of from about 1 micron to about 10 microns.

21. The nonwoven fabric laminate of claim 16 wherein the meltblown fibers have an average diameter in the range of up to about 8 microns and the spunbonded fibers have an average diameter in the range of from about 8 microns to about 30 microns.

22. The nonwoven fabric laminate of claim 16 wherein the first spunbonded layer, the meltblown layer and the second spunbonded layer are intermittently bonded to form the nonwoven fabric laminate.

23. The nonwoven fabric laminate of claim 16 having a SAM retention level of greater than 95 percent using the SAM Shake Test.

24. The nonwoven fabric laminate of claim 16 having a SAM retention level of greater than 98 percent using the SAM Shake Test.
